(19) United States
(12) Patent Application Publication
(10) Pub. No.: US 2008/0300636 A1
(43) Pub. Date: Dec. 4, 2008

(54) Fracture reduction instrument for osseous body

(76) Inventors: Olivier Carli, Guyancourt (FR); Hugues Mousselard, Paris (FR); Pascal Rokegem, Pasteur (FR)

Correspondence Address: CLARK & BRODY, 1090 VERMONT AVENUE, NW, SUITE 250, WASHINGTON, DC, 20005, US

(21) Appl. No.: 12/068,799

(22) Filed: Feb. 12, 2008

Related U.S. Application Data
(60) Provisional application No. 60/924,432, filed on May 15, 2007.

(30) Foreign Application Priority Data
Feb. 14, 2007 (FR) 07 53250

(51) Int. Cl. A61B 17/56; A61B 17/70; A61F 5/00

(52) U.S. Cl. 606/280; 606/60; 606/86 B

(57) ABSTRACT

The invention relates to a fracture reduction instrument for an osseous body, extending between a distal end (2) and a proximal end, the distal end comprising at least one deformable element (5) suitable for passing from a relaxed position for positioning of the distal end inside the osseous body, to a deformed position for forming a cavity inside the osseous body, the deformable element being stressed by a shift mechanism (6) so that the deformable element may pass from one position to another and vice versa. According to the invention, the deformable element (5) is a blade whereof the two ends are provided with axes of rotation (7, 8) mounted freely in rotation between two assembly supports (9, 10) whereof at least one is stressed by the shift mechanism (6) to be mobile in translation so as to bring together the ends of the blade (5) by rotation of said ends.

The present invention relates to the technical field of surgical instrumentation for treating osseous body fractures.

The object of the invention relates precisely to instrumentation and a method for reducing vertebral fractures.

In the area of the spine, reduction of a vertebra fracture requires restoring the initial shape of the vertebra before proceeding with filling it using cement or an osseous substitute adapted to confer to the vertebra sufficient resistance to support loads in compression.

Various solutions in the prior art have been put forward for reducing vertebral fractures. For example, an instrument is known which extends between a proximal end and a distal end equipped with an inflatable cuff suitable for passing from a deflated position to allow it to be positioned inside the vertebra, to an inflated position so as to push back the upper vertebral plate and restore localised lordosis.

This instrument does not respond to expectations by surgeons of patients. In fact, it has noted that expansion of the cuff is not controllable. In reality, during the inflation operation of the cuff, expansion of the cuff appears mainly in the zones of least resistance. It follows that the direction of reduction of the fracture cannot be controlled.

In an attempt to rectify these disadvantages U.S. Pat. No. 6,676,665 describes a fracture reduction instrument comprising a rod enclosed by a tube provided at its distal end with several axial cut-outs delimiting elastically deformable arms. The rod and the tube are fixes at their distal ends and can be shifted in the opposite direction so as to deploy arms.

Similarly, US patent application 2002/0026197 describes a fracture reduction instrument comprising a deformable tubular element comprising axial cut-outs for delimiting elastically deformable arms. This tubular deformable element is placed between a first stop borne by a sliding rod mounted inside a tube and a second stop arranged on the tube. The rod is shifted relative to the tube to ensure deployment of the arms.

The advantage of such instruments is to have preferential support zones in directions determined so as to push back the osseous body. It proves however that such instruments do not develop sufficient thrust effort such that they have no significant support point so as to push back, on a large scale, the upper plate of a vertebra.

The object of the invention is thus to remedy the disadvantages of the prior art by proposing a novel reduction instrument for fractures of osseous bodies, adapted to exert a support effort controlled in direction, according to a value sufficient for pushing back an osseous body efficaciously and on a large scale.

To attain such an objective, the object of the invention relates to a fracture reduction instrument for osseous body extending between a distal end and a proximal end, the distal end comprising at least one deformable element suitable for passing from a position relaxed for the positioning of the distal end inside the osseous body to a deformed position to form a cavity inside the osseous body, the deformable element being stressed by a shift mechanism so that the deformable element may pass from one position to another and vice versa.

According to the invention, the deformable element is a blade whereof the two ends are provided with axes of rotation mounted freely in rotation between two assembly supports whereof at least one is stressed by the shift mechanism to be mobile in translation so as to bring together the ends of the blade by rotation of said ends.

According to an embodiment the deformable blade is mounted removably on the assembly supports.

According to an embodiment, the deformable blade comprises an elastically deformable central part, connected on either side to the axes of rotation by junction arms.

Advantageously, the deformable blade has a predefined shape to permit elastic deformation by buckling towards the exterior of the instrument.

For example, when the blade is in the relaxed position, the central part extends according to an axis offset relative to an axis passing through the two axes of rotation.

It should be noted that the central part of the deformable blade has a profile symmetrical or dissymmetrical relative to the mediator plane of the two axes of rotation.

According to another characteristic of the invention, the junction arms exhibit greater rigidity than the rigidity of the central part of the deformable blade.

According to an embodiment, the axes of rotation protrude laterally on either side of the junction arms, to cooperate with the assembly supports.

For example, the instrument comprises:
a tube provided at its distal end with an assembly opening of the blade and a support assembly for an axis of rotation of the blade.
and with a rod mounted to slide inside the tube and provided at its distal end with a support assembly for the other axis of rotation of the blade.

Thus, each assembly support is delimited by oblong holes arranged in the tube of the rod and extending radially to take up the axes of rotation, the oblong holes communicating with a passage foramen d'un junction arm according to its thickness.

In this example, the sliding rod is part of the shift mechanism of the screw-nut type.

Various other characteristics will merge from the following description in reference to the attached diagrams which show, by way of non-limiting examples, embodiments of the object of the invention.

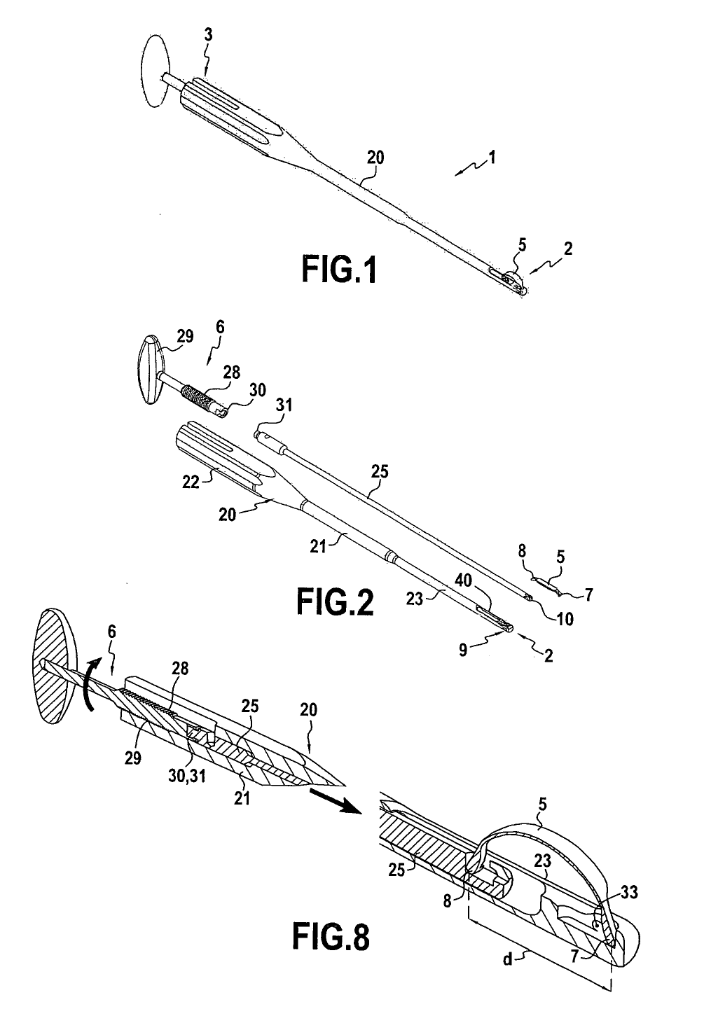

FIG. 1 is a general view of an embodiment of a reduction instrument according to the invention.

FIG. 2 is an exploded view of the diverse pieces making up a reduction instrument according to the invention.

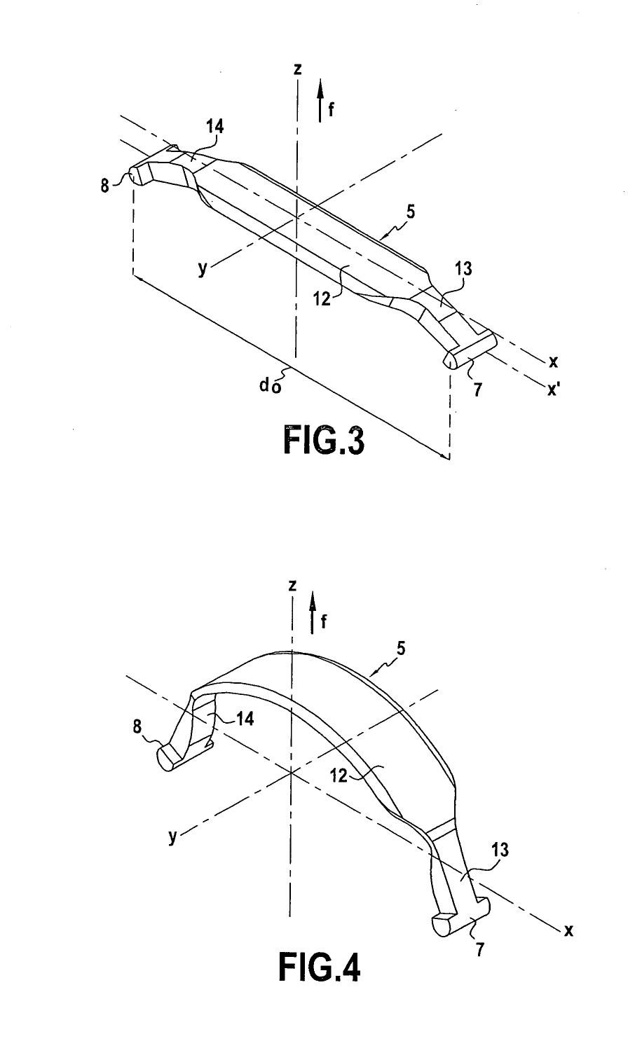

FIGS. 3 and 4 are views respectively in a relaxed position and in a deformed position of a deformable blade used in the reduction instrument according to the invention.

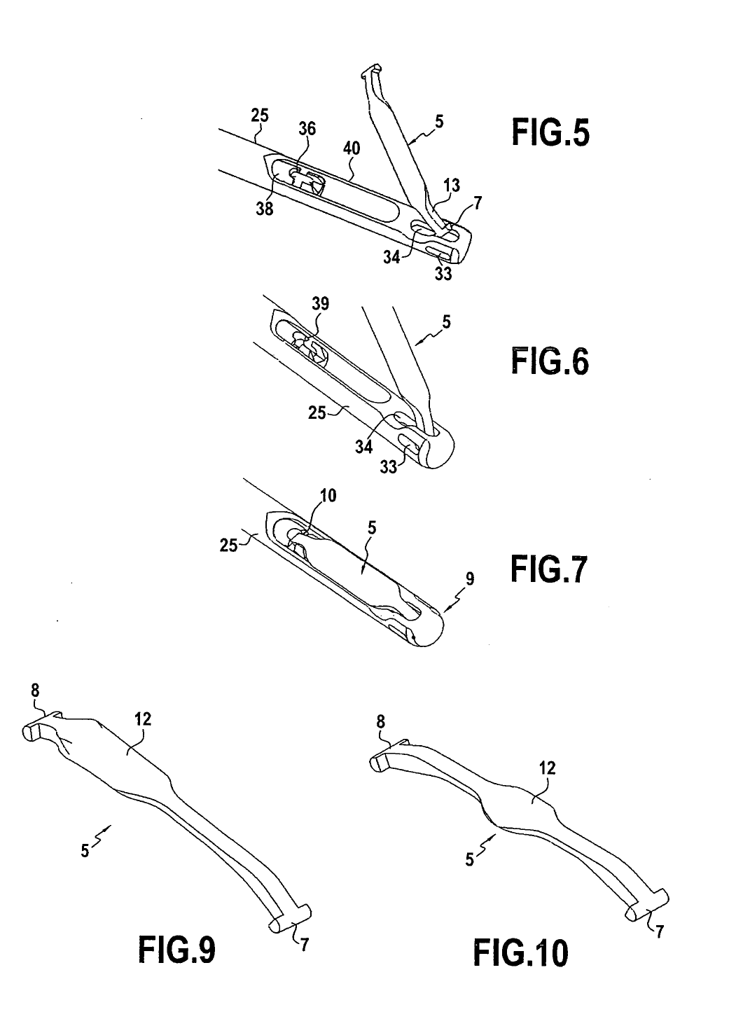

FIGS. 5 to 7 are views illustrating the assembly of the deformable blade on a reduction instrument according to the invention.

FIG. 8 is a view showing the operating principle of the reduction instrument according to the invention.

FIGS. 9 and 10 are views of two variant embodiments of a deformable blade according to the invention.

As will be specified from FIGS. 1 and 2, the object of the invention relates to a fracture reduction instrument 1 for an osseous body in the direction general. The reduction instrument 1 is particularly adapted for reducing fractures of vertebrae so as to push back the upper plate of the vertebras and restore localised lordosis.

The reduction instrument 1 is in the form of an elongated body extending between a distal end 2 and a proximal end 3. The reduction instrument 1 comprises at least one deformable element 5 suitable for passing from a relaxed position for positioning of the distal end 2 inside the osseous body (FIG. 3) to a deformed position to form a cavity inside the osseous body (FIGS. 1, 4 and 8). This deformable element 5 is stressed by a shift mechanism 6 so that the deformable element can pass from one position to another and vice versa.

In keeping with the invention, the deformable element 5 is a blade whereof the two ends are provided with axes of rotation 7 and 8 mounted freely in rotation between two assembly supports respectively 9 and 10. One of the assembly supports, specifically 10 in the example illustrated, is stressed by the shift mechanism 6 to be mobile in translation so as to be able to bring together the two ends of the blade by rotation of said ends.

As emerges more precisely from FIGS. 3 and 4, the deformable blade 5 comprises an elastically deformable central part 12 connected on either side to the axes of rotation 7 and 8, by junction arms 13 and 14 respectively. The elastically deformable central part 12 extends in a relaxed position substantially in a plane defined by the longitudinal axis x and the transversal axis y. This deformable part 12 has a flattened rectangular shape and a width taken according to the transversal axis y, greater than the width of the junction arms 13, 14.

According to a characteristic of the invention, the junction arms 13 and 14 exhibit rigidity greater than the rigidity of the central part 12 of the deformable blade. In other terms the junction arms 13 and 14 are considered as being undeformable compared to the elastically deformable central part 12.

According to a preferred embodiment characteristic, the deformable blade 5 has a predefined shape to produce elastic deformation by buckling towards the exterior of the instrument 1. As emerges more precisely from FIG. 3, in a relaxed position of the blade 5 the central part 12 extends according to a longitudinal axis x which is offset relative to an axis x′ passing through the two axes of rotation 7 and 8. In other terms, the axes of rotation 7 and 8 do not extend in the extension in the same plane as the central part 12 of the deformable blade. In a relaxed position of the deformable blade 5 the junction arms 13 and 14 extend slightly inclined relative to the plane x, y of the central part 12. The deformable blade 5 thus has a convex profile turned according to a direction of the direction z extending perpendicularly to the plane xy. In other terms, the blade 5 is adapted such that deformation by buckling occurs systematically in the direction f of the direction z with relative coming together of the axes of rotation 7 and 8.

According to an advantageous characteristic of the object of the invention, the deformable blade 5 is mounted removably on the assembly supports 9 and 10. The deformable blade 5 can thus be changed after each deformation by buckling.

Deformation by buckling of the blade 5 is obtained by relative coming together of the axes of rotation 7 and 8 resulting from translation of one and/or of the other of the ends of the deformable blade 5. In the example illustrated in the diagrams, deformation by buckling is obtained by the shift in translation of the axis of rotation 8 relative to the axis of rotation 7. Of course, it could be envisaged to flex the blade while bringing together its two ends.

According to the embodiment illustrated, the assembly support 9 is arranged at the distal end of a tube 20 arranged to delimit a pedicular extra part 21 prolonged towards the proximal end 3 by a sleeve 22 and prolonged towards the distal end 2 by an intrapedicular part 23. The intrapedicular part 23 of the tube has a diameter less than or equal to 5 mm since it is designed to be introduced to the pedicle of a vertebra.

The assembly support 10 is arranged at the distal end of a rod 25 mounted to slide inside the tube 20. This rod 25 is shifted in translation by means of the shift mechanism 6. In the illustrated example, the shift mechanism 6 is of the screw-nut type. The shift mechanism 6 comprises, in the illustrated example, a threaded rod 28 cooperating with a tapping 29 arranged in the tube 20 at the level of the sleeve 22. The threaded rod 28 is provided at its protruding end of the tube 20 with a grip 29 and at its opposite end with an assembly system 30 having a complementary part 31 presented by the proximal end of the rod 25 such that rotation of the threaded rod 28 in a direction or an opposite direction results in exerting thrust or traction effort on the rod 25. In this way, rotation of the grip 29 in a clockwise or anticlockwise direction ensures that the rod 25 slides inside the tube 20 in one direction or the other. Of course, the shift mechanism 6 can be different to a screw-nut system.

FIGS. 5 to 8 explain a preferred embodiment of the assembly supports 9 and 10. The assembly support 9 is delimited by two oblong holes 33 arranged in the tube 25 radially or diametrically opposed. These two oblong holes 33 communicate with a passage foramen 34 arranged axially on the tube 25. This foramen 34 has width adapted to allow passage of the junction arm 13 according to its thickness and a length adapted for placing the axis of rotation 7 in the oblong holes 33, as will be described further on in the description.

Similarly, the assembly support 10 is delimited by two oblong holes 36 arranged in the rod 25 extending radially or diametrically opposite. These oblong holes 36 communicate with a passage foramen 38 for the junction arm 14. This foramen 38 has a width adapted to ensure passage of the junction arm 14 according to its width. This foramen 38 is provided with two passages 39 for the axis of rotation 8, terminating in the oblong holes 36, outside the proximal part of the oblong holes 36 such that the axis of rotation 8 is blocked in the plane yz when stopped against the proximal part of the oblong holes 36.

The distal end 2 of the tube 20 comprises an opening 40 for assembly of the blade arranged substantially in the extension of the foramen 34 to allow accessibility to the distal end of the sliding rod 25.

The oblong holes 33, 36 are intended to take up the axes of rotation 7 and 8. For this purpose, each axis of rotation 7 and 8 protrudes laterally on either side of the junction arms 13 and 14.

Placing a deformable blade 5 according to the invention derives directly from the preceding description. The blade 5 is introduced inside the foramen 34 by its junction arm 13 turned according to its thickness (FIG. 5). The deformable blade 5 is pivoted so as to guide the axis of rotation 7 into penetrating the oblong holes 33 as shown in FIG. 6. In this position, the axis of rotation 7 can slide and turn freely inside the oblong holes 33 but is blocked in the plane yz. The sliding rod 25 is placed such that its distal end is accessible via the opening 40. In this position, the deformable blade 5 is folded over such that the axis of rotation 8 is inserted into the two oblong holes 36 after having passed through the passage opening 40, the foramen 38 and the passages 39. The rod 25 is moved towards the distal end of the tube 20 such that on one hand the axis of rotation 8 comes into contact with the proximal part of the oblong holes 36, and on the other hand the axis of rotation 7 is supported on the distal part of the oblong holes 33. In this position, the blade 5 occupies a relaxed assembly position, at the same time assembled at these two ends since the axes of rotation 7, 8 are blocked in displacement in the plane yz, preventing extraction of the blade.

A shift in the sliding rod 25 in the direction of the rapprochement of the distal end of the rod 25 towards the distal end of the tube 20 results in shortening of the distance d0 between the axes 7 and 8 of the blade to attain a distance d (FIG. 8). The axes of rotation 7 and 8 of the blade come together by turning thus favouring buckling of the blade 5. It should be considered that buckling of the blade 5 results in rotation of the axes of rotation 7, 8 thus limiting restrictions on the blade 5. The reduction instrument 1 according to the invention thus produces by means of the deformable blade 5 a support point developing considerable stress over a relatively substantial range.

The reduction instrument 1 according to the invention thus produces a deployment range of up to 12 mm. Also, the flex of the deformable blade 5 can be adapted for developing efforts of variable values. For example, the blade can have variable flexes for developing efforts of up to 110 newtons. Of course, the deformable blade can have different shapes as a function of the need for restitution of the vertebral plate.

In the example illustrated in FIGS. 3 and 4, the central part of the deformable blade has a symmetrical profile relative to the mediator plane of the two axes of rotation 7 and 8. Of course, a dissymmetrical shape such as illustrated in FIG. 9 can be provided. According to this variant, the central part 12 is not centred relative to the transversal axis y passing through the middle of the axes of rotation 7, 8. FIG. 10 illustrates another embodiment in which the central part 12 of the blade is limited according to the axis x relative to the solution illustrated in FIGS. 3 and 4 where this central part 12 extends over the majority of the blade.

The deformable blade 5 can be made from various materials. For example, it can be made from metallic materials (TA6V, chrome cobalt, nitinol super elastic) or from polymers or composites (PEEK, PEEK/CARBONE).

The advantage of the reduction instrument 1 according to the present invention is to be able to direct reduction of the fracture in the third plane specifically:
the axial plane in the limit of the displacement permitted by the pedicle,
the sagittal plane by adjusting penetration of the reduction instrument,
the frontal plane by rotation of the instrument for example for reducing the side walls.

The reduction instrument 1 is thus orientable so as to select a support point on the vertebral plate in the lowest zone. The instrument rests on the pedicle to constitute counter-support reaction to the efforts undertaken for reduction. The shift mechanism can flex the blade 5 which on contact of the plate will lift the latter to effect reductions. The metal blade 5 flexes under the action of the shortening mechanism of the distance do from the distance d. A lever action can also aid reduction to the extent that it can support the pedicle.

The invention is not limited to the examples described and illustrated as various modifications can be made without departing from its scope.

1. A reduction instrument for fractures of osseous bodies, extending between a distal end (2) and a proximal end (3), the distal end comprising at least one deformable element (5) suitable for passing from one relaxed position for positioning the distal end inside the osseous body, to a deformed position to form a cavity inside the osseous body, the deformable element being stressed by a shift mechanism (6) so that the deformable element can pass from one position to another and vice versa, characterised in that the deformable element (5) is a blade the two ends of which are provided with axes of rotation (7, 8) mounted freely in rotation between two assembly supports (9, 10) whereof at least one is stressed by the shift mechanism (6) to be mobile in translation so as to bring together the ends of the blade (5) with rotation of said ends.

2. The reduction instrument as claimed in claim 1, characterised in that the deformable blade (5) is mounted removably on the assembly supports (9, 10).

3. The reduction instrument as claimed in claim 1, characterised in that the deformable blade (5) comprises an elastically deformable central part (12), connected on either side to the axes of rotation by junction arm.

4. The reduction instrument as claimed in claim 3, characterised in that the deformable blade (5) has predefined shape to produce elastic deformation by buckling towards the exterior of the instrument.

5. The reduction instrument as claimed in claim 4, characterised in that in the relaxed position of the blade (5), the central part (12) extends according to an axis offset relative to an axis passing through the two axes of rotation (7, 8).

6. The reduction instrument as claimed in claim 3, characterised in that the central part (12) of the deformable blade has a symmetrical or dissymmetrical profile relative to the mediator plane of the two axes of rotation.

7. The reduction instrument as claimed in claim 4, characterised in that the junction arms (13, 14) exhibit rigidity greater than the rigidity of the central part (12) of the deformable blade.

8. The reduction instrument as claimed in claim 1, characterised in that the axes of rotation (7, 8) protrude laterally on either side of the junction arm to cooperate with the assembly supports (9, 10).

9. The reduction instrument as claimed in claim 8, characterised in that it comprises:
a tube (20) provided at its distal end with an opening (40) for mounting the blade and an assembly support for an axis of rotation of the blade,
and a rod (25) mounted to slide inside the tube and provided at its distal end with an assembly support (10) for the other axis of rotation of the blade.

10. The reduction instrument as claimed in claim 9, characterised in that each assembly support (9, 10) is delimited by oblong holes (33, 36) arranged in the tube of the rod and extending radially to take up the axes of rotation (7, 8), the oblong holes (33, 36) communicating with a passage foramen (34, 38) of a junction arm (13, 14) according to its thickness.

11. The reduction instrument as claimed in claim 9, characterised in that the sliding rod (25) is part of the shift mechanism (6) of the screw-nut type.

12. The reduction instrument as claimed in claim 2, characterised in that the deformable blade (5) comprises an elastically deformable central part (12), connected on either side to the axes of rotation by junction arm.

13. The reduction instrument as claimed in claim 4, characterised in that the central part (12) of the deformable blade has a symmetrical or dissymmetrical profile relative to the mediator plane of the two axes of rotation.

14. The reduction instrument as claimed in claim 5, characterised in that the central part (12) of the deformable blade has a symmetrical or dissymmetrical profile relative to the mediator plane of the two axes of rotation.
